(19) United States
(12) Patent Application Publication
(10) Pub. No.: US 2022/0351299 A1
(43) Pub. Date: Nov. 3, 2022

(54) INFORMATION PROCESSING APPARATUS, METHOD, AND NON-TRANSITORY COMPUTER READABLE MEDIUM

(71) Applicant: TOYOTA JIDOSHA KABUSHIKI KAISHA, Aichi-ken (JP)

(72) Inventors: Tadahiro KASHIWAI, Nagoya-shi (JP); Toyokazu NAKASHIMA, Nagoya-shi (JP); Hideo HASEGAWA, Nagoya-shi (JP); Kazuya IWATA, Nagoya-shi (JP); Shinichiro IIDA, Toyota-shi (JP); Kiyonori YOSHIDA, Toyota-shi (JP); Yota SAKURAI, Nagoya-shi (JP)

(21) Appl. No.: 17/661,025

(22) Filed: Apr. 27, 2022

(30) Foreign Application Priority Data
Apr. 28, 2021 (JP) 2021-076639

(51) Int. Cl. G06Q 40/08; G06Q 30/02; G16H 20/30

(52) U.S. Cl. CPC G06Q 40/08; G06Q 30/0207; G16H 20/30

(57) ABSTRACT

A controller of an information processing apparatus acquires travel information on a travel by a user from a departure point to a destination, and determines to grant an incentive to the user in a case in which the user is determined to have performed a travel on foot in addition to a travel using a vehicle based on the acquired travel information.

### CROSS-REFERENCE TO RELATED APPLICATION

This application claims priority to Japanese Patent Application No. 2021-076639, filed on Apr. 28, 2021, the entire contents of which are incorporated herein by reference.

### TECHNICAL FIELD

The present disclosure relates to an information processing apparatus, a method, and a program.

### BACKGROUND

Patent Literature (PTL) 1 discloses an information processing system that prompts use of park and ride.

### CITATION LIST

### Patent Literature

PTL 1: JP 2018-081022 A

### SUMMARY

Park and ride is a system that prompts travels using public transportation after travels using private vehicles. However, it is demanded to prompt travels on foot after travels using vehicles to improve the health of users.

It would be helpful to prompt travels on foot and improve the health of users.

An information processing apparatus according to an embodiment of the present disclosure includes a controller configured to:

acquire travel information on a travel by a user from a departure point to a destination; and

determine to grant an incentive to the user in a case in which the user is determined to have performed a travel on foot in addition to a travel using a vehicle based on the acquired travel information.

A method according to an embodiment of the present disclosure is a method performed by an information processing apparatus, the method including:

acquiring travel information on a travel by a user from a departure point to a destination; and

determining to grant an incentive to the user in a case in which the user is determined to have performed a travel on foot in addition to a travel using a vehicle based on the acquired travel information.

A program according to an embodiment of the present disclosure is configured to cause a computer to execute operations, the operations including:

acquiring travel information on a travel by a user from a departure point to a destination; and

determining to grant an incentive to the user in a case in which the user is determined to have performed a travel on foot in addition to a travel using a vehicle based on the acquired travel information.

According to an embodiment of the present disclosure, it is possible to prompt travels on foot and improve the health of users.

### BRIEF DESCRIPTION OF THE DRAWINGS

In the accompanying drawings:

FIG. 1 is a block diagram illustrating a schematic configuration of a system according to an embodiment of the present disclosure;

FIG. 2 is a block diagram illustrating a schematic configuration of an information processing apparatus according to an embodiment of the present disclosure;

FIG. 3 is a block diagram illustrating a schematic configuration of a terminal apparatus according to an embodiment of the present disclosure;

FIG. 4 is a flowchart illustrating operations of an information processing apparatus according to an embodiment of the present disclosure; and

FIG. 5 is a diagram illustrating an example of granting an incentive according to an embodiment of the present disclosure.

### DETAILED DESCRIPTION

Hereinafter, an embodiment of the present disclosure will be described.

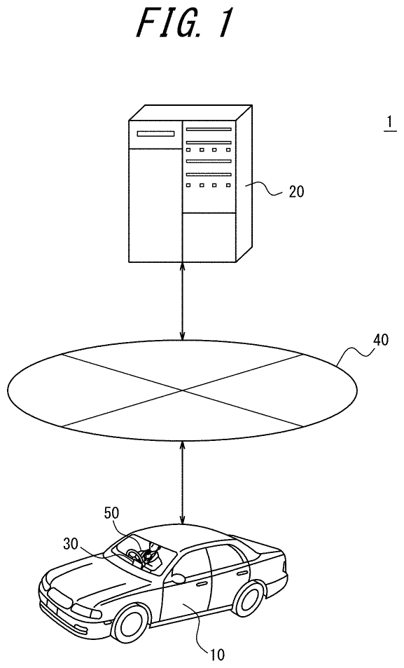

An outline of a system 1 according to the embodiment of the present disclosure will be described with reference to FIG. 1.

The system 1 includes a vehicle 10, an information processing apparatus 20, and a terminal apparatus 30. The vehicle 10, the information processing apparatus 20, and the terminal apparatus 30 are communicably connected to a network 40.

The vehicle 10 is an automobile, for example, but is not limited to this, and may be any appropriate vehicle. The automobile is, for example, a gasoline vehicle, a battery electric vehicle (BEV), a hybrid electric vehicle (HEV), a plug-in hybrid electric vehicle (PHEV), a fuel cell electric vehicle (FCEV), or the like, but is not limited to these. The vehicle 10 may be driven by a driver, or the driving may be automated at any level. The automation level is, for example, any one of Level 1 to Level 5 according to the level classification defined by Society of Automotive Engineers (SAE). The vehicle 10 may be a Mobility as a Service (MaaS) dedicated vehicle.

The information processing apparatus 20 is a computer such as a server that belongs to a cloud computing system or another type of computing system.

The terminal apparatus 30 is connected to the vehicle 10 as an external device or is held by a user 50. The terminal apparatus 30 is, for example, a mobile device such as a mobile phone, a smartphone, or a tablet. Alternatively, the terminal apparatus 30 is mounted in the vehicle 10 and is used by a user 50 who is on board the vehicle 10. The terminal apparatus 30 may be, for example, an in-vehicle device such as a car navigation device.

The network 40 includes the Internet, at least one wide area network (WAN), at least one metropolitan area network (MAN), or any combination thereof. The network 40 may include at least one wireless network, at least one optical network, or any combination thereof. The wireless network is, for example, an ad hoc network, a cellular network, a wireless local area network (LAN), a satellite communication network, or a terrestrial microwave network.

An outline of the present embodiment will be described with reference to FIG. 1.

A controller 21 of the information processing apparatus 20 acquires travel information on a travel by a user 50 from a departure point to a destination. The controller 21 of the information processing apparatus 20 determines to grant an incentive to the user 50 in a case in which the user 50 is determined to have performed a travel on foot in addition to a travel using the vehicle 10 based on the acquired travel information.

Thus, according to the present embodiment, the user 50 is granted an incentive in a case in which the user 50 has performed a travel on foot in addition to a travel using the vehicle 10. Accordingly, it is possible to prompt travels on foot and improve the health of the user 50.

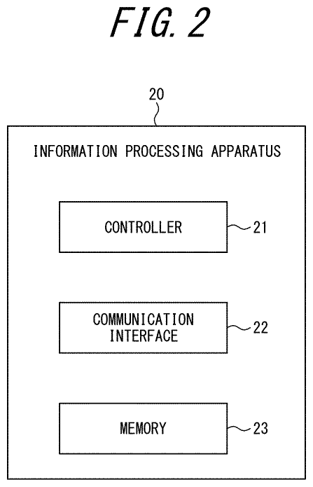

A configuration of the information processing apparatus 20 according to the present embodiment will be described with reference to FIG. 2.

The information processing apparatus 20 includes the controller 21, a communication interface 22, and a memory 23.

The controller 21 includes at least one processor, at least one programmable circuit, at least one dedicated circuit, or any combination thereof. The processor is a general purpose processor such as a central processing unit (CPU) or a graphics processing unit (GPU), or a dedicated processor that is dedicated to specific processing. The programmable circuit is, for example, a field-programmable gate array (FPGA). The dedicated circuit is, for example, an application specific integrated circuit (ASIC). The controller 21 executes processes related to operations of the information processing apparatus 20 while controlling components of the information processing apparatus 20.

The communication interface 22 includes at least one interface for communication. The interface for communication is compliant with, for example, a mobile communication standard, a wired LAN standard, or a wireless LAN standard, but not limited to these, and may be compliant with any communication standard. The communication interface 22 receives data to be used for the operations of the information processing apparatus 20, and transmits data obtained by the operations of the information processing apparatus 20.

The memory 23 includes at least one semiconductor memory, at least one magnetic memory, at least one optical memory, or any combination thereof. The semiconductor memory is, for example, random access memory (RAM) or read only memory (ROM). The RAM is, for example, static random access memory (SRAM) or dynamic random access memory (DRAM). The ROM is, for example, electrically erasable programmable read only memory (EEPROM). The memory 23 functions as, for example, a main memory, an auxiliary memory, or a cache memory. The memory 23 stores data to be used for the operations of the information processing apparatus 20 and data obtained by the operations of the information processing apparatus 20. In the present embodiment, the data to be used for the operations of the information processing apparatus 20 includes a system program, an application program, a database, map information, and the like.

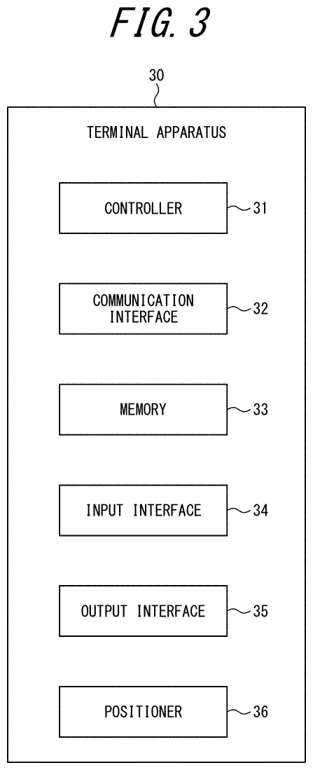

A configuration of the terminal apparatus 30 according to the present embodiment will be described with reference to FIG. 3.

The terminal apparatus 30 includes a controller 31, a communication interface 32, a memory 33, an input interface 34, an output interface 35, and a positioner 36.

The controller 31 includes at least one processor, at least one programmable circuit, at least one dedicated circuit, or any combination thereof. The processor is a general purpose processor such as a CPU or a GPU, or a dedicated processor that is dedicated to specific processing. The programmable circuit is, for example, an FPGA. The dedicated circuit is, for example, an ASIC. The controller 31 executes processes related to operations of the terminal apparatus 30 while controlling components of the terminal apparatus 30.

The communication interface 32 includes at least one interface for communication. The interface for communication is, for example, an interface compliant with a mobile communication standard such as a Long Term Evolution (LTE), the 4th generation (4G) standard, or the 5th generation (5G) standard, an interface compliant with a short-range wireless communication standard such as Bluetooth® (Bluetooth is a registered trademark in Japan, other countries, or both), or a LAN interface. The communication interface 32 receives data to be used for the operations of the terminal apparatus 30, and transmits data obtained by the operations of the terminal apparatus 30.

The memory 33 includes at least one semiconductor memory, at least one magnetic memory, at least one optical memory, or any combination thereof. The semiconductor memory is, for example, RAM or ROM. The RAM is, for example, SRAM or DRAM. The ROM is, for example, EEPROM. The memory 33 functions as, for example, a main memory, an auxiliary memory, or a cache memory. The memory 33 stores data to be used for the operations of the terminal apparatus 30 and data obtained by the operations of the terminal apparatus 30.

The input interface 34 includes at least one interface for input. The interface for input is, for example, a physical key, a capacitive key, a pointing device, a touch screen integrally provided with a display, a camera, a light detection and ranging or laser imaging, detection, and ranging (LiDAR) sensor, or a microphone. The input interface 34 accepts an operation for inputting data to be used for the operations of the terminal apparatus 30. The input interface 34, instead of being included in the terminal apparatus 30, may be connected to the terminal apparatus 30 as an external input device. As an interface for connection, for example, an interface compliant with a standard such as Universal Serial Bus (USB), HDMI® (HDMI is a registered trademark in Japan, other countries, or both), or Bluetooth® can be used.

The output interface 35 includes at least one interface for output. The interface for output is, for example, a display or a speaker. The display is, for example, a liquid crystal display (LCD) or an organic electro-luminescent (EL) display. The output interface 35 outputs data obtained by the operations of the terminal apparatus 30. The output interface 35, instead of being included in the terminal apparatus 30, may be connected to the terminal apparatus 30 as an external output device. As an interface for connection, for example, an interface compliant with a standard such as USB, HDMI®, or Bluetooth® can be used.

The positioner 36 includes at least one global navigation satellite system (GNSS) receiver. GNSS is, for example, Global Positioning System (GPS), Quasi-Zenith Satellite System (QZSS), BeiDou Navigation Satellite System (BDS), Global Navigation Satellite System (GLONASS), or Galileo. The positioner 36 measures the position of the terminal apparatus 30. The positioner 36 may include any speed sensor and gyro sensor.

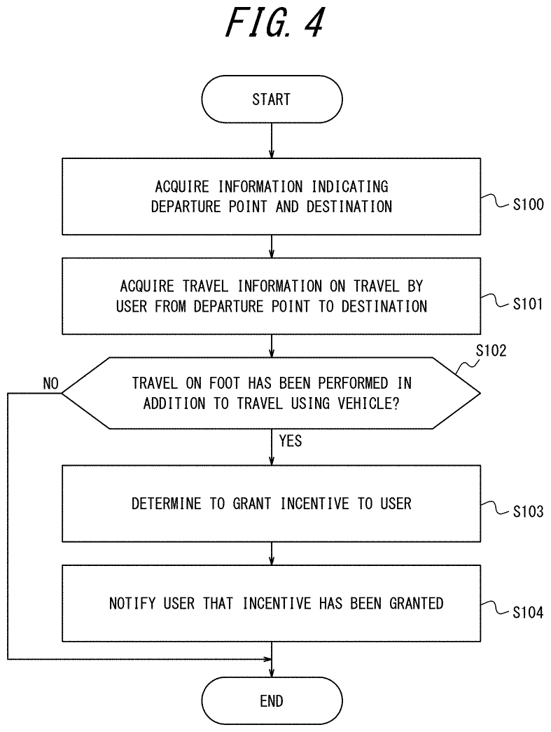

Operations of the information processing apparatus 20 according to the present embodiment will be described with reference to FIG. 4. These operations correspond to a method according to the present embodiment.

In the present embodiment, the terminal apparatus 30 is assumed to be connected to the vehicle 10 as an external device when a user 50 is traveling using the vehicle 10, and held by the user 50 when the user 50 is traveling on foot. Accordingly, the position of the terminal apparatus 30 is assumed to correspond to the positions of the vehicle 10 and the user 50 when the user 50 is traveling using the vehicle 10, and correspond to the position of the user 50 when the user 50 is traveling on foot.

Step S100: The controller 21 of the information processing apparatus 20 acquires information indicating a departure point and information indicating a destination.

Specifically, the controller 31 of the terminal apparatus 30 accepts an operation for inputting a destination via the input interface 34 by the user 50 to a map application running on the terminal apparatus 30. The controller 31 of the terminal apparatus 30 transmits, via the communication interface 32, information indicating the destination input to the map application running on the terminal apparatus 30 to the information processing apparatus 20. The controller 21 of the information processing apparatus 20 receives the information indicating the destination from the terminal apparatus 30 via the communication interface 22. A departure point may be automatically detected by the positioner 36 of the terminal apparatus 30, or may be manually input by the user 50 similarly to the destination. In either case, the controller 31 of the terminal apparatus 30 transmits information indicating the departure point to the information processing apparatus 20 via the communication interface 32. The controller 21 of the information processing apparatus 20 receives the information indicating the departure point from the terminal apparatus 30 via the communication interface 22.

Step S101: The controller 21 of the information processing apparatus 20 acquires travel information on a travel by the user 50 from the departure point to the destination.

Specifically, the positioner 36 of the terminal apparatus 30 repeatedly measures positions of the terminal apparatus 30 until a position of the terminal apparatus 30 matches the destination. The controller 31 of the terminal apparatus 30 transmits information indicating a position of the terminal apparatus 30 and information indicating a measurement time to the information processing apparatus 20 via the communication interface 32 each time a measurement is made by the positioner 36. The controller 21 of the information processing apparatus 20 receives, via the communication interface 22, the information indicating the position of the terminal apparatus 30 and the information indicating the measurement time from the terminal apparatus 30. Upon determining that the position of the terminal apparatus 30 matches the destination, the controller 21 of the information processing apparatus 20 acquires a travel route and travel speeds of the terminal apparatus 30 that correspond to the travel information on the travel by the user 50, by analyzing the temporal transition of positions of the terminal apparatus 30 until the position of the terminal apparatus 30 matches the destination. A match in the present embodiment is not necessarily a mathematically strict match, and may be a match within an allowable error range.

Step S102: The controller 21 of the information processing apparatus 20 determines whether the user 50 has performed a travel on foot in addition to a travel using the vehicle 10 based on the travel information acquired in step S101. The processing proceeds to step S103 in a case in which the user 50 is determined to have performed a travel on foot in addition to a travel using the vehicle 10. On the other hand, the processing ends in a case in which the user 50 is determined not to have performed a travel on foot in addition to a travel using the vehicle 10.

Specifically, the controller 21 of the information processing apparatus 20 determines whether a section where a travel speed of the terminal apparatus 30 matches a general walking speed (hereinafter, also referred to as a “travel section where a travel on foot has been performed”) is included in the travel route of the terminal apparatus 30 acquired in step S101, in addition to a section where a travel speed of the terminal apparatus 30 matches a general vehicle speed (hereinafter, also referred to as a “travel section where a travel using the vehicle 10 has been performed”). The controller 21 of the information processing apparatus 20 determines that the user 50 has performed a travel on foot in addition to a travel using the vehicle 10 in a case in which a travel section where a travel on foot has been performed is determined to be included in addition to a travel section where a travel using the vehicle 10 has been performed. The controller 21 of the information processing apparatus 20 stores in the memory 23 information indicating the travel section where the travel using the vehicle 10 has been performed and information indicating the travel section where the travel on foot has been performed. The controller 21 of the information processing apparatus 20 may identify the travel section where the travel using the vehicle 10 has been performed based on a driving distance, a driving route, or the like acquired from the vehicle 10. The controller 21 of the information processing apparatus 20 may also identify the travel section where the travel using the vehicle 10 has been performed or the travel section where the travel on foot has been performed based on vibrations or the like, acquired from the terminal apparatus 30, generated due to the vehicle 10 driving or the user 50 walking.

Here, step S102 will be described more specifically using an example in which the departure point is the home of the user 50 and the destination is a facility such as a shopping center. This example assumes that after the user 50 travels using the vehicle 10 from the home to a parking lot located around the facility, the user 50 parks the vehicle 10 in a parking space within the parking lot and then travels on foot to an entrance of the facility. In this case, the controller 21 of the information processing apparatus 20 determines that a travel using the vehicle 10 has been performed from the home to the parking space in which the vehicle 10 is parked. The controller 21 of the information processing apparatus 20 also determines that a travel on foot has been performed from the parking space in which the vehicle 10 is parked to the entrance of the facility. The parking lot located around the facility may include a parking lot attached to the facility, or may include a parking lot that is not attached to the facility and is located at a location within walking distance (e.g., within 10 minutes on foot) of the facility.

The travel section where the travel on foot has been performed is not necessarily a section for which a travel on foot is recommended by, for example, a car navigation device or the like, and in a case in which the user 50 has traveled on foot through a partial section included in a section for which a travel using the vehicle 10 is recommended by the car navigation device or the like, may be the partial section.

Step S103: The controller 21 of the information processing apparatus 20 determines to grant an incentive to the user 50.

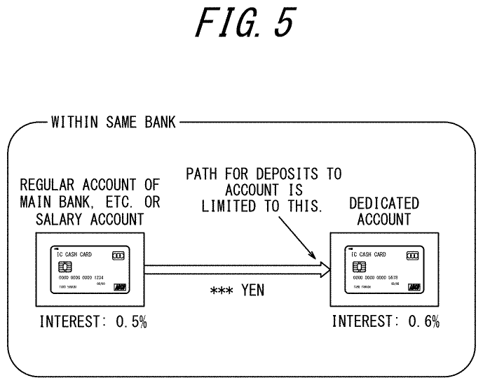

Specifically, the controller 21 of the information processing apparatus 20 determines to grant to the user 50 an incentive including granting points that can be used when the user 50 purchases some product or receives some service, raising an interest rate on a deposit account, or lowering a commission on investment such as securities. An example of granting an incentive to the user 50 by raising an interest rate on a deposit account will be described with reference to FIG. 5. In the example of FIG. 5, a preset amount of money is transferred from a bank account regularly used by the user 50 to a dedicated account the interest rate on which is higher than the bank account. However, this is applied on condition that the balance in the bank account regularly used by the user 50 be more than the preset amount.

In step S103, the controller 21 of the information processing apparatus 20 may determine an incentive to be granted to the user 50 according to the travel distance of the travel on foot. Specifically, the controller 21 of the information processing apparatus 20 calculates the travel distance of the travel on foot based on information indicating the travel route of the travel on foot with reference to the memory 23. The controller 21 of the information processing apparatus 20 determines to grant more incentives to the user 50 as the calculated travel distance of the travel on foot increases. For example, in a case in which a facility such as a shopping center is the destination, the travel distance of a travel on foot from a parking space in which the vehicle 10 is parked to an entrance of the facility increases as the user 50 parks the vehicle 10 in a parking space located at a location farther from the entrance of the facility. Accordingly, the controller 21 of the information processing apparatus 20 determines to grant more incentives to the user 50 when the vehicle 10 is parked in a parking space located at a location far from the entrance of the facility. This enables prompting travels on foot and thus the health of the user 50 can be improved.

In step S103, the controller 21 of the information processing apparatus 20 may determine an incentive to be granted to the user 50 according to one or more attributes of the user 50. Specifically, the controller 21 of the information processing apparatus 20 acquires, via the communication interface 22, information indicating attributes of the user 50 including age, a Body Mass Index (BMI), or a disease history of the user 50 from a health management application or the like running on the terminal apparatus 30. The controller 21 of the information processing apparatus 20 estimates a health risk of the user 50 based on the acquired information indicating the attributes of the user 50. For example, the controller 21 of the information processing apparatus 20 estimates that the health risk of the user 50 is higher as the age of the user 50 increases. For example, the controller 21 of the information processing apparatus 20 estimates that the health risk of the user 50 is higher as the BMI of the user 50 increases. For example, the controller 21 of the information processing apparatus 20 estimates that the health risk of the user 50 is higher as the user 50 has experienced more diseases. The controller 21 of the information processing apparatus 20 determines to grant more incentives to the user 50 as the estimated health risk of the user 50 increases. This enables prompting the user 50 with a high health risk to perform travels on foot and thus the health of the user 50 can be improved. The health risk may include a risk of developing hypertension, diabetes, heart disease, or the like, but is not limited to these.

In step S103, the controller 21 of the information processing apparatus 20 may determine an incentive to be granted to the user 50 according to a travel record of past travels on foot by the user 50. Specifically, each time a travel from a departure point to a destination is performed, the controller 21 of the information processing apparatus 20 adds the travel distance of the latest travel on foot to cumulative travel distance that corresponds to the travel record of past travels on foot by the user 50, thereby updating the cumulative travel distance, and stores the cumulative travel distance in the memory 23. Then, in step S103, the controller 21 of the information processing apparatus 20 determines, with reference to the memory 23, to grant more incentives as the cumulative travel distance increases. This enables prompting the user 50 to continue travels on foot and thus the health of the user 50 can be improved.

Step S104: The controller 21 of the information processing apparatus 20 notifies the user 50 that an incentive has been granted via the communication interface 22.

Specifically, the controller 21 of the information processing apparatus 20 generates a message indicating that an incentive has been granted. The controller 21 of the information processing apparatus 20 transmits the generated message to the terminal apparatus 30 via the communication interface 22. The controller 31 of the terminal apparatus 30 receives the transmitted message from the information processing apparatus 20 via the communication interface 32. The controller 31 of the terminal apparatus 30 outputs the received message to the user 50 through screen display, audio, or the like, via the output interface 35.

As described above, the controller 21 of the information processing apparatus 20 according to the present embodiment acquires travel information on a travel by a user 50 from a departure point to a destination. The controller 21 of the information processing apparatus 20 determines to grant an incentive to the user 50 in a case in which the user 50 is determined to have performed a travel on foot in addition to a travel using the vehicle 10 based on the acquired travel information.

According to such a configuration, the user 50 is granted an incentive in a case in which the user 50 has performed a travel on foot in addition to a travel using the vehicle 10. Accordingly, it is possible to prompt travels on foot and improve the health of the user 50.

While the present disclosure has been described with reference to the drawings and examples, it should be noted that various modifications and revisions may be implemented by those skilled in the art based on the present disclosure. Accordingly, such modifications and revisions are included within the scope of the present disclosure. For example, functions or the like included in each component, each step, or the like can be rearranged without logical inconsistency, and a plurality of components, steps, or the like can be combined into one or divided.

As a variation, an embodiment in which the configuration and operations of the information processing apparatus 20 are distributed to multiple computers capable of communicating with each other can be implemented. For example, an embodiment in which some or all of the components of the information processing apparatus 20 are provided in the vehicle 10 can also be implemented. For example, a car navigation device mounted in the vehicle 10 may include some or all of the components of the information processing apparatus 20.

As a variation, in step S103 described above, the controller 21 of the information processing apparatus 20 may determine to grant an incentive to the user 50 with respect to a life insurance plan. Specifically, the controller 21 of the information processing apparatus 20 estimates a health condition of the user 50 based on a travel record of past travels on foot by the user 50. The controller 21 of the information processing apparatus 20 proposes a life insurance plan suitable for the user 50 based on the estimated health condition of the user 50. For example, the controller 21 of the information processing apparatus 20 estimates that the health condition of the user 50 is better as the user 50 has performed more travels on foot in the past. The controller 21 of the information processing apparatus 20 proposes a life insurance plan with a lower insurance premium from among a plurality of life insurance plans stored in advance in the memory 23 as the user 50 has a better health condition.

As a variation, the controller 21 of the information processing apparatus 20 may determine an incentive to be granted to the user 50 according to the height difference of a route on which the user 50 has traveled on foot. Specifically, the controller 21 of the information processing apparatus 20 calculates the height difference of a travel section where a travel on foot has been performed with reference to the memory 23. The controller 21 of the information processing apparatus 20 determines to grant more incentives to the user 50 as the calculated height difference increases. For example, in a case in which the user 50 has parked the vehicle 10 in a parking lot located at a location within walking distance of a facility such as a shopping center and then gone uphill on foot to travel to an entrance of the facility, the user 50 is granted more incentives.

As a variation, an embodiment in which a general purpose computer functions as the information processing apparatus 20 according to the embodiment described above can also be implemented. Specifically, a program in which processes for realizing the functions of the information processing apparatus 20 according to the embodiment described above are written is stored in a memory of a general purpose computer, and the program is read and executed by a processor. Accordingly, the present disclosure can also be implemented as a program executable by a processor, or a non-transitory computer readable medium storing the program.

1. An information processing apparatus comprising a controller configured to:
acquire travel information on a travel by a user from a departure point to a destination; and
determine to grant an incentive to the user in a case in which the user is determined to have performed a travel on foot in addition to a travel using a vehicle based on the acquired travel information.

2. The information processing apparatus according to claim 1, wherein the travel on foot includes a travel on foot from a parking space in which the vehicle is parked, within a parking lot located around the destination, to the destination.

3. The information processing apparatus according to claim 1, wherein the controller is configured to determine the incentive according to a travel distance of the travel on foot.

4. The information processing apparatus according to claim 1, wherein the controller is configured to determine the incentive according to one or more attributes of the user.

5. The information processing apparatus according to claim 4, wherein the one or more attributes includes age, a BMI, or a disease history.

6. The information processing apparatus according to claim 1, wherein the controller is configured to determine the incentive according to a travel record of past travels on foot by the user.

7. The information processing apparatus according to claim 6, wherein the controller is configured to estimate a health condition of the user based on the travel record and propose a life insurance plan suitable for the user based on the estimated health condition.

8. A method performed by an information processing apparatus, the method comprising:
acquiring travel information on a travel by a user from a departure point to a destination; and
determining to grant an incentive to the user in a case in which the user is determined to have performed a travel on foot in addition to a travel using a vehicle based on the acquired travel information.

9. The method according to claim 8, wherein the travel on foot includes a travel on foot from a parking space in which the vehicle is parked, within a parking lot located around the destination, to the destination.

10. The method according to claim 8, further comprising determining the incentive according to a travel distance of the travel on foot.

11. The method according to claim 8, further comprising determining the incentive according to one or more attributes of the user.

12. The method according to claim 11, wherein the one or more attributes includes age, a BMI, or a disease history.

13. The method according to claim 8, further comprising determining the incentive according to a travel record of past travels on foot by the user.

14. The method according to claim 13, further comprising:
estimating a health condition of the user based on the travel record; and
proposing a life insurance plan suitable for the user based on the estimated health condition.

15. A non-transitory computer readable medium storing a program configured to cause a computer to execute operations, the operations comprising:
acquiring travel information on a travel by a user from a departure point to a destination; and
determining to grant an incentive to the user in a case in which the user is determined to have performed a travel on foot in addition to a travel using a vehicle based on the acquired travel information.

16. The non-transitory computer readable medium according to claim 15, wherein the travel on foot includes a travel on foot from a parking space in which the vehicle is parked, within a parking lot located around the destination, to the destination.

17. The non-transitory computer readable medium according to claim 15, wherein the operations further comprise determining the incentive according to a travel distance of the travel on foot.

18. The non-transitory computer readable medium according to claim 15, wherein the operations further comprise determining the incentive according to one or more attributes of the user.

19. The non-transitory computer readable medium according to claim 15, wherein the operations further comprise determining the incentive according to a travel record of past travels on foot by the user.

20. The non-transitory computer readable medium according to claim 19, wherein the operations further comprise:
estimating a health condition of the user based on the travel record; and
proposing a life insurance plan suitable for the user based on the estimated health condition.
